SEQUENCE LISTING (12 sequences)

SEQ ID NO: 1 - DNA, 18 bp, Artificial Sequence
  primer
gaagtcgactgcgctccc

SEQ ID NO: 2 - DNA, 19 bp, Artificial Sequence
  primer
actccgccctataagcatc

SEQ ID NO: 3 - DNA, 21 bp, Artificial Sequence
  primer
gtcaccacccaaatccttatt

SEQ ID NO: 4 - DNA, 18 bp, Artificial Sequence
  primer
ggcgatcttgaacgtgat

SEQ ID NO: 5 - DNA, 21 bp, Artificial Sequence
  primer
aagatcagcaaacgcaaacac

SEQ ID NO: 6 - DNA, 21 bp, Artificial Sequence
  primer
cccgtaccaacctggataagt

SEQ ID NO: 7 - DNA, 24 bp, Artificial Sequence
  primer
ggggatgtggaatttttatacaag

SEQ ID NO: 8 - DNA, 23 bp, Artificial Sequence
  primer
cgccaggacaaaccagtatgtag

SEQ ID NO: 9 - DNA, 19 bp, Artificial Sequence
  primer
gtgaaaaggaccccacgaa

SEQ ID NO: 10 - DNA, 24 bp, Artificial Sequence
  primer
agtcaagggcatatcctacaacaa

SEQ ID NO: 11 - DNA, 19 bp, Artificial Sequence
  primer
caacgggtacaaacgagtc

SEQ ID NO: 12 - DNA, 21 bp, Artificial Sequence
  primer
cttccttggcttcaaccttag

(19) United States
(12) Patent Application Publication
(10) Pub. No.: US 2009/0318413 A1
(43) Pub. Date: Dec. 24, 2009

(54) BRONCHIAL SMOOTH MUSCLE REMODELING INVOLVES CALCIUM-DEPENDENT ENHANCED MITOCHONDRIAL BIOGENESIS IN ASTHMA

(75) Inventors: Patrick BERGER, Merignac (FR); Thomas Trian, Newtown (AU); Rodrigue Rossignol, Merignac (FR); Roger Marthan, Bordeaux (FR); J. Manuel Tunon de Lara, Bordeaux (FR); Pierre-Olivier Girodet, Bordeaux (FR)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(73) Assignee: UNIVERSITE VICTOR SEGALEN BORDEAUX 2, BORDEAUX (FR)

(21) Appl. No.: 12/487,273

(22) Filed: Jun. 18, 2009

Related U.S. Application Data
(60) Provisional application No. 61/073,677, filed on Jun. 18, 2008.

(51) Int. Cl. A61K 31/554; A61K 31/137; A61K 31/4422

(52) U.S. Cl. 514/211.07; 514/654; 514/356

(57) ABSTRACT

The present invention thus provides a method of inhibiting bronchial smooth muscle remodeling in asthma, comprising the step of administering to a subject having asthma an agent that inhibits calcium-dependent mitochondrial biogenesis.

This application claims benefit of U.S. Provisional Application No. 61/073,677 filed Jun. 18, 2008, the entire disclosure of which is incorporated by reference herein in its entirety.

Throughout this application, various references or publications are cited. Disclosures of these references or publications in their entireties are hereby incorporated by reference into this application in order to more fully describe the state of the art to which this invention pertains.

### FIELD OF THE INVENTION

This invention relates to the field of treatment for asthma.

### BACKGROUND OF THE INVENTION

Asthma and chronic obstructive pulmonary disease (COPD) are inflammatory airway diseases that are characterized by different patterns of airway remodeling (1). Nevertheless, the decrease in lung function that characterizes both diseases is associated with an increased mass of bronchial smooth muscle (BSM) (2, 3), which is likely to be the most important abnormality responsible for the airway narrowing observed in response to bronchoconstricting stimuli (4). The mechanisms underlying such remodeling of smooth muscle remain largely unknown.

On the one hand, in smooth muscle from asthmatic patients, excessive in vitro proliferation of BSM cells has been demonstrated (5-7). In addition, decreased apoptosis of BSM cells has also been demonstrated, although this was in a rat model of experimental asthma (7). On the other hand, in COPD, smooth muscle remodeling appears limited to airways distal to the fourth generation (3, 8). Although an increase in TGF-α1 production by BSM cells has been proposed, such localization and the complete mechanism remain unexplained (9). Whatever its cause, BSM remodeling is poorly sensitive to current therapeutics in both asthma and COPD.

Mitochondria play a major role in both cell proliferation and apoptosis (10, 11). In cancer, for instance, targeting of mitochondrial function and mitochondrial anti-apoptotic protein bcl-2 has been used to either suppress the proliferation of tumor cells (10) or induce cell apoptosis in solid tumors (12). Mitochondria are also involved in other diseases, such as neuron-degenerative diseases (11). However, their role in asthma or COPD remains to be investigated.

It was discovered that asthmatic BSM was characterized by an increased mitochondrial biogenesis that, in turn, enhanced cell proliferation. Mitochondria were identified as new target for the treatment of asthmatic smooth muscle remodeling. The Applicant has developed drugs interacting with mitochondrial biogenesis, including drugs acting at the site of calcium homeostasis, for preventing and/or reversing BSM remodeling in asthma.

### SUMMARY OF THE INVENTION

The present invention thus provides a method of inhibiting bronchial smooth muscle remodeling in asthma, comprising the step of administering to a subject having asthma an agent that inhibits calcium-dependent mitochondrial biogenesis. In one embodiment, the agent inhibits calcium influx in bronchial smooth muscle cells. In another embodiment, the agent inhibits proliferation of bronchial smooth muscle cells. In yet another embodiment, the agent is a calcium channel blocker. Examples of calcium channel blocker include, but are not limited to, gallopamil verapamil, devapamil, emopamil, nifedipine, nicardipine, diltiazem and salt thereof.

The present invention also provides a method of inhibiting bronchial smooth muscle remodeling in asthma, comprising the step of administering to a subject having asthma an agent that inhibits cellular signaling of calcium-dependent mitochondrial biogenesis. In one embodiment, the agent inhibits expression or function of signaling molecule involved in calcium-dependent mitochondrial biogenesis. Examples of signaling molecule include, but are not limited to, mitochondrial transcription factor A, nuclear respiratory factor-1, peroxisome proliferatorαactivated receptor α coactivator—1α, and calcium/calmodulin-dependent protein kinase IV. In one embodiment, the expression or function of the signaling molecule can be inhibited at the protein, DNA or RNA level.

The present invention also provides a pharmaceutical composition for inhibiting bronchial smooth muscle remodeling in asthma, comprising a therapeutically active amount of an agent that inhibits calcium-dependent mitochondrial biogenesis in asthmatic bronchial smooth muscle cells and a pharmaceutically acceptable vehicle. According to this embodiment, the agent is present and administered in an amount sufficient to inhibit calcium influx in asthmatic bronchial smooth muscle cells. Alternatively, the amount of agent is present and administered in an amount sufficient to inhibit cellular signaling of calcium-dependent mitochondrial biogenesis. In one embodiment, the agent inhibits expression or function of signaling molecule involved in calcium-dependent mitochondrial biogenesis. Examples of signaling molecule include, but are not limited to, mitochondrial transcription factor A, nuclear respiratory factor-1, peroxisome proliferator-activated receptor coactivator-1α, and calcium/calmodulin-dependent protein kinase IV. In one embodiment, the expression or function of the signaling molecule can be inhibited at the protein, DNA or RNA level.

### BRIEF DESCRIPTION OF THE DRAWINGS

FIG. 1 shows BSM remodeling in both asthma and COPD. Representative optic microscopic images from bronchial sections stained with HES were obtained from an asthmatic (A), a COPD (B), or a control subject (C) and observed at 200× magnification. Smooth muscles were visualized (SM). Bars, 50 μm. The normalized smooth muscle area was assessed from microscopic images (D). Bronchial specimens were obtained from asthmatic (black column; n=10), COPD (gray column; n=7), and control subjects (white column; n=6). Data are the mean±the SEM. *, P<0.05 between populations using ANOVA with the use of Bonferroni's test.

FIG. 2 shows increased mitochondrial mass and activity in asthmatic BSM. Representative electronic microscopic images from bronchial sections were obtained from an asthmatic (A), a COPD (B), or a control subject (C) and observed at 26,000× magnification. Some smooth muscle mitochondria were visualized (arrows). Bars, 0.2 μm. The number (D) and the density (E) of mitochondria were assessed from electronic microscopic images (n=4 for each population). Mitochondrial mass was assessed by the porin content using Western blot (F) (n=8 for asthmatics, n=5 for COPD, and n=7 for controls). Endogenous cellular oxygen consumption was evaluated by oxygraphy (G) (n=5 for asthmatics, n=4 for COPD, and n=4 for controls). BSM cells (BSMC) were obtained from asthmatic (black columns), COPD (gray columns), and control subjects (white columns). Data are the mean±the SEM. *, P<0.05 between populations using ANOVA with the use of Bonferroni's test.

FIG. 3 shows increased mitochondrial biogenesis in asthmatic BSM. Representative confocal images of the mitochondrial network after three dimensional reconstruction were obtained from asthmatic (A), COPD (B), or control (C) BSM cells. Bars, 10 μm. mtTFA, NRF-1, and PGC-1μ levels were assessed by both Western blot (D) and quantitative RT-PCR (E). BSM cells were obtained from asthmatic (black columns; n=6), COPD (gray columns; n=7), and control subjects (white columns; n=6). Data are the mean±the SEM. *, P<0.05 between populations using ANOVA with the use of Bonferroni's test.

FIG. 4 shows altered cell calcium homeostasis in asthmatic BSM. Phosphorylated CaMK-IV (P-CaMK-IV) levels were assessed by Western blot (A). Representative intracellular calcium responses after stimulation for 30 s by 10⁻⁵M acetylcholine (ACh) are presented in BSM cells from asthmatic (B), COPD (C), or control subjects (D). As a reference, response from the control cell (D) is presented as a gray line (B and C). The area under the curve was assessed from the calcium response (E). BSM cells were analyzed in the absence (−) or presence (+) of 2 mM extracellular Ca²⁺ or 1 μM methoxyverapamil (gallopamil or D600). Cells were obtained from asthmatic (black columns; n=5), COPD (gray columns; n=4), and control subjects (white columns; n=4). Data are the mean±the SEM. *, P<0.05 between populations within an experimental condition using ANOVA with the use of Bonferroni's test. †, P<0.05 between experimental conditions versus 2 mM Ca²⁺ without D600 within a population using paired Student's t tests.

FIG. 5 shows the effect of methoxyverapamil (gallopamil or D600) on mitochondrial biogenesis and content. PGC-1α(A), NRF-1 (B), mtTFA (C), and porin (D) levels were assessed by Western blot in BSM cells cultured in the absence (−) or presence (+) of 1 μM D600 for 48 h. Cells were obtained from asthmatic (black columns; n=5), COPD (gray columns; n=4), and control subjects (white columns; n=4). Data are the mean±the SEM. *, P<0.05 between populations within an experimental condition using ANOVA with the use of Bonferroni's test. †, P<0.05 between the absence and the presence of D600 within a population using paired Student's t tests

FIG. 6 shows asthmatic BSM cell proliferation is mitochondria dependent. BSM cell proliferation curves were obtained using either glucose (A) or galactose (B) in the culture medium. The doubling times of cell growth (C) were obtained from the proliferation curves. (D) BrdU incorporations were measured. BSM cells were cultured in various experimental conditions, i.e., glucose, galactose, glucose+ethidium bromide (Et Br), or glucose+cyclic GMP (cGMP). BSM cells were obtained from asthmatic (black symbols and columns; n=4), COPD (gray symbols and columns; n=4), and control subjects (white symbols and columns; n=4). Data are the mean±the SEM. *, P<0.05 between populations within an experimental condition using ANOVA with the use of Bonferroni's test. †, P<0.05 between experimental conditions versus glucose within a population using paired Student's t tests.

FIG. 7 shows the effect of ethidium bromide and cyclic GMP on the porin content. Mitochondrial mass was assessed by the porin content using Western blot. BSM cells were obtained from asthmatic (black columns; n=4), COPD (gray columns; n=4), and control subjects (white columns; n=4) and were cultured in the absence (−) or presence (+) of ethidium bromide (Et Br) or cyclic GMP (cGMP) for 6 d before the experiments. Data are the mean±the SEM. *, P<0.05 between populations within an experimental condition using ANOVA with the use of Bonferroni's test. †, P<0.05 between experimental conditions versus glucose within a population using paired Student's t tests.

FIG. 8 shows the effect of methoxyverapamil (gallopamil or D600) on BSM cell proliferation. BSM cell proliferation was measured using BrdU incorporations. Cells were cultured in the absence (−) or presence (+) of 1 μM D600 for 48 h. BSM cells were obtained from asthmatic (black columns; n=4), COPD (gray columns; n=4), and control subjects (white columns; n=4). Data are the mean±the SEM. *, P<0.05 between populations within an experimental condition using ANOVA with the use of Bonferroni's test. †, P<0.05 between the absence and the presence of D600 within a population using paired Student's t tests.

### DETAILED DESCRIPTION OF THE INVENTION

The present invention provides a method of inhibiting bronchial smooth muscle remodeling in asthma, comprising the step of administering to a subject having asthma a therapeutically active amount of an agent that inhibits calcium-dependent mitochondrial biogenesis. The present invention also provides a pharmaceutical composition for inhibiting bronchial smooth muscle remodeling in asthma comprising a therapeutically effective amount of an agent that inhibits calcium-dependent mitochondrial biogenesis and a pharmaceutically acceptable vehicle.

In one embodiment, the agent inhibits calcium influx in bronchial smooth muscle cells or inhibits proliferation of bronchial smooth muscle cells. In another embodiment, the agent is a calcium channel blocker. Examples of calcium channel blocker include, but are not limited to, gallopamil verapamil, devapamil, emopamil, anipamil, nifedipine, nicardipine, diltiazem, or salts thereof. Currently, 3 classes of calcium channel blockers in multiple immediate or sustained-release preparations are available in the United States. These classes are as follows: phenylalkylamines (e.g., gallopamil, verapamil), benzothiazines (e.g. diltiazem), and dihydropyridines (e.g. nifedipine, amlodipine). Calcium channel blockers function by binding to the L-subtype, voltage-sensitive, slow calcium channels in cell membranes, thereby decreasing the flow of calcium into the cell. Each calcium channel blocker has a certain degree of tissue specificity, but they do have common properties. They are all absorbed early in the GI system, are substantially bound by plasma proteins, and are predominantly metabolized by the liver.

The pharmaceutical compositions according to the present invention include the formulations that are suitable for any desired administration, including but not limited to, oral, parenteral, including subcutaneous, intradermal, intramuscular, intravenous and intraarticular, rectal and topical, including dermal, buccal, sublingual and intraocular administration, or by inhalation via a dose inhaler or nebulization.

The present invention also provides a method of inhibiting bronchial smooth muscle remodeling in asthma, comprising the step of administering to a subject having asthma an agent that inhibits cellular signaling of calcium-dependent mitochondrial biogenesis. The present invention also provides a pharmaceutical composition for inhibiting bronchial smooth muscle remodeling in asthma comprising a therapeutically effective amount of an agent that inhibits cellular signaling of calcium-dependent mitochondrial biogenesis and a pharmaceutically acceptable vehicle.

In one embodiment, the agent inhibits expression or function of signaling molecule involved in calcium-dependent mitochondrial biogenesis. Examples of signaling molecule include, but are not limited to, mitochondrial transcription factor A, nuclear respiratory factor-1, peroxisome proliferator-activated receptor coactivator-1α, and calcium/calmodulin-dependent protein kinase IV. In one embodiment, the expression or function of the signaling molecule can be inhibited at the protein, DNA or RNA level.

The present invention also provides a method of treating asthma comprising the step of administering to a subject having asthma a therapeutically effective dose of an agent that inhibits calcium-dependent mitochondrial biogenesis in asthmatic bronchial smooth muscle cells. The treated subject can be a patient, preferably a human patient, with severe asthma or non severe asthma (where remodeling of smooth muscle has just started). In one embodiment, the agent that inhibits calcium-dependent mitochondrial biogenesis is a calcium channel blocker such as gallopamil verapamil, devapamil, emopamil, anipamil, nifedipine, nicardipine, diltiazem, or salts thereof.

A therapeutically effective dose or amount of an agent is intended to mean an amount or a proportion thereof sufficient to inhibit calcium influx in bronchial smooth muscle cells and/or to inhibit proliferation of bronchial smooth muscle cells, and/or inhibit cellular signaling of calcium-dependent mitochondrial biogenesis, and/or inhibit expression or function of signaling molecule involved in calcium-dependent mitochondrial biogenesis.

The agent as described above may be administered to the subject via various following routes of administration, such as oral, parenteral, including subcutaneous, intradermal, intramuscular, or intravenous, rectal and topical, including dermal, buccal, sublingual or by inhalation.

While it may be possible for the agents of the invention to be administered as the raw chemical, it is preferable to provide them as a pharmaceutical composition, for any of the disorders described herein. According to a further aspect, the present invention provides a pharmaceutical composition comprising a therapeutically effective amount of the agent of the invention or a pharmaceutically acceptable salt, together with one or more pharmaceutically acceptable vehicle thereof and optionally one or more other therapeutic ingredients for indications such as asthma.

One of ordinary skill in the art would readily determine a suitable treatment schedule. In one embodiment, a subject or patient can be treated with 70 mg to 150 mg or 100 mg tablets of a calcium channel blocker such as gallopamil twice a day for 12 months, or 6 months, 8 months, 10 months, 14 months, or up to 24 months. According to the IUPAC designation, gallopamil corresponds to 5-[2-(3,4-dimémethoxyphenyl)ethyl-methylamino]-2-propan-2-yl-2-(3,4,5-triméthoxyphenyl)pentanenitrile hydrochloride, and is known under ATC code C08DA02. Also, gallopamil is marketed under PROCORUM® by Abbott, e.g., 50 mg or 100 mg tablets, wherein the active substance according to the DCI designation is gallopamil hydrochloride or methoxyverapamil or D600. Such tablets comprise microcrystalline cellulose, sodium alginate, povidone, magnesium stearate, and purified water, as excipients.

In a preferred embodiment, the calcium channel blocker such as gallopamil is administered in a sufficient therapeutic amount to obtain (i) 5-50%, 5-40%, 5-30%, 5-20%, or 5-15% decrease of asthmatic smooth muscle remodeling; or (ii) 5-50%, 5-40%, 5-30%, 5-20%, or 5-15% decrease of bronchial thickness assessed by 3D analysis of computed tomography; or (iii) a control of asthma according to the questionnaire (ACQ) validated by Juniper; or (iv) a reduction of BSM mitochondrial number and activity assessed by electronic microscopy, oxygraphy, or Western blot.

In another preferred embodiment, treatment with an agent that inhibits calcium-dependent mitochondrial biogenesis as described above can be combined with standard care therapy for asthma. For example, one standard therapy for asthma comprises 2 treatments: (1) inhaled corticosteroids such as beclomethasone (2000 pg/day equivalent), or budesonide (64 μg/dose) marketed under Rhinocort®, or fluticasone, and (2) LABA (Long acting beta 2 agonists) (200 μg/day equivalent), such as formoterol or salmeterol, and optionally a third one which is an oral corticoid such as prednisolone.

The present invention is based on the discovery that both asthma and COPD are by BSM remodeling, a specific mitochondria-dependent pathway is required for BSM proliferation only in asthma. This pathway is initiated by an altered calcium homeostasis, upon the activation of asthmatic BSM cells. Proliferation of BSM in both health and COPD was found to be, at the very least, less mitochondria dependent. The Applicant has thus characterized mitochondria as being a specific new therapeutic target in airway remodeling in asthma.

Special attention was paid when comparing data from severe asthmatics to that of COPD patients because both diseases have been shown to present smooth muscle remodeling (2, 3, 17). Using a variety of different experimental approaches, it was discovered that asthmatic BSM expressed a higher number of active mitochondria and a clear aspect of intense mitochondrial biogenesis. The Applicant has then assessed mitochondrial mass using various parameters, including the number of mitochondria by electron microscopy both ex vivo and in vitro, the mitochondrial network by confocal microscopy, and the porin content by Western blot. All of these methods have provided consistent results. Significant correlations were also found between in vitro BSM porin content, which was a relevant quantitative estimate of mitochondrial mass, and both the duration of the disease and the FEV₁/FVC ratio within the asthmatic population. On the one hand, the longer the duration of the disease that was known to favor airway remodeling (18), the higher the mitochondrial mass, and on the other hand, the lower the FEV₁/FVC ratio, which also reflected airway remodeling, the higher the mitochondrial mass. Moreover, to obtain a comprehensive assessment of mitochondrial content, the expression level of mtTFA was measured by both quantitative RT-PCR and Western blot to assess mitochondrial respiratory chain content, as mitochondrial overall content and respiratory chain content were not always linked (19).

The increased number of mitochondria was limited to asthmatic BSM cells compared with endothelial and epithelial cells from asthmatic, COPD, or control subjects. However, two characteristics of such asthmatic populations deserve further comment. First, the Applicant has found that it was unlikely that asthma treatments interfered with the observed changes because, on the one hand, 9/17 COPD patients took treatments similar to that of asthmatics, and, on the other hand, no difference has been found between controls and COPD. Second, the younger mean age of the asthmatic population was unlikely to contribute to this increase in mitochondrial content because (a) this increase persisted when comparing asthmatics to a subgroup of nonsmoking controls whose mean age was similar, and (b) in skeletal muscle, if not any other muscle, age does not influence mitochondrial content (20). Such mitochondrial characteristics in any type of smooth muscle cells with potential pathophysiological implications was never previously characterized and described. It has been previously reported that in a variety of differentiated tissues, a mitochondrial dysfunction increases mitochondrial biogenesis, suggesting a cellular compensatory mechanism (21). However, no mitochondrial dysfunction was reported in the case of asthmatic patient because the respiration of asthmatic BSM mitochondria was efficiently coupled. Similarly, artificial activation of mitochondrial biogenesis of mouse neonatal cardiac myocytes largely induced a coupled respiration (22).

Many factors may control mitochondrial biogenesis. PGC-1α is a well-known master activator of mitochondrial biogenesis through the production of both NRF-1 and mtTFA in various cell types, including myoblast (23), fibroblast, or adipocytes (24). To this regard, the Applicant has demonstrated that such a cascade was activated in asthmatic BSM cells, as shown by the concomitant up-regulation of PGC-1α, NRF-1, and mtTFA. Moreover, cyclic GMP has been shown to activate PGC-1α and mitochondrial biogenesis in various cell lines, including U937, L6, and PC12 (16, 25). It was observed that cyclic GMP induces mitochondrial biogenesis in both control and COPD BSM cells, but not in asthmatic BSM cells. These findings suggested that mitochondrial biogenesis in asthmatic BSM cells may already be up-regulated. In this connection, cyclic GMP also improved cell proliferation of control and COPD BSM cells, but not those of asthmatics. It could be argued that cyclic GMP regulates many different genes in smooth muscle, such as vascular smooth muscle (26). However, cyclic GMP has been shown to inhibit, rather than enhance, the proliferation of vascular smooth muscle cells, as well as that of mesangial cells and various fibroblasts (26). In addition, direct improvement of mitochondrial biogenesis by transgenic overexpression of PGC-1α activated skeletal muscle atrophy (27) and cardiac muscle dysfunction (22). Conversely, cyclic GMP increased the proliferation of endothelial cells, but the role of mitochondria in this phenomenon has never been so far (28).

PGC-1α activation can be calcium-dependent (29). The Applicant has discovered that CaMK-IV was more phosphorylated in asthmatic BSM cells than in both controls and COPD. Rises in calcium concentration have been previously shown to activate CaMK-IV in other cell types, including skeletal muscle cells (29) or osteoclasts (30). Interestingly, the Applicant has demonstrated that calcium homeostasis in asthmatic BSM cells was altered, thus providing a mechanistic explanation for the increased activation of calcium-dependent signaling enzymes such as CaMK-IV (31). A gallopamil (D600) sensitive calcium influx was suspected to account for such asthma-induced alteration in calcium homeostasis. The Applicant evidenced that this calcium influx was the initial priming event because, when blocked, mitochondrial biogenesis and subsequent asthmatic BSM cell-increased proliferation was inhibited. Thus blockage of such influx was predicted to be beneficial. Previous studies using methoxyverapamil (gallopamil) were focused on short-term effects for up to 4 wk and did not show airway remodeling (32, 33).

Finally, mitochondria-deficient BSM cells were generated by depletion of mitochondrial DNA with ethidium bromide, which is a potent inhibitor of mitochondrial DNA replication and transcription (10, 15). Mitochondria-deficient BSM cells from asthmatics were unable to proliferate, thereby confirming the importance of mitochondria in asthmatic BSM cell proliferation. Thus, the increased mitochondrial biogenesis observed in asthmatic BSM cells appeared to be a cause rather than a consequence of the asthmatic BSM cell increased proliferation. However, it was well known that training can increase mitochondrial biogenesis in skeletal muscle (34). It was unlikely that a similar phenomenon appears to the BSM from asthmatics. In this study, none of the asthmatics presented recent exacerbations, and all of these patients were treated by relaxant 2 agonists.

As described herein above, the pharmaceutical compositions according to the present invention include the formulations that are suitable for any desired administration, including but not limited to, oral, parenteral, including subcutaneous, intradermal, intramuscular, intravenous and intraarticular, rectal and topical, including dermal, buccal, sublingual and intraocular administration, or by inhalation via a dose inhaler or nebulization.

The formulations may conveniently be presented in unit dosage form and may be prepared by any of the methods well known in the art of pharmacy. All methods include the step of bringing into association an agent of the invention or a pharmaceutically acceptable salt with the carrier or vehicle which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules or tablets each containing a predetermined amount of the agent as described above; as a powder or granules; as a solution or a suspension in an aqueous liquid or a non-aqueous liquid; or as an oil-in-water liquid emulsion or a water-in-oil liquid emulsion. The active agent may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active agent therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampuls and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

The invention being generally described, will be more readily understood by reference to the following examples which are included merely for purposes of illustration of certain aspects and embodiments of the present invention, and are not intended to limit the invention.

### EXAMPLE 1

### Clinical Populations

The clinical characteristics of all subjects are shown in Table 1. All of the 14 severe persistent asthmatics were lifelong nonsmokers and received stable treatments, including oral or inhaled corticosteroids and β32 agonists. Ten of them were atopic. The 17 moderate to severe chronic obstructive pulmonary disease (COPD) patients were either current or former smokers, and 9 of them received stable treatments, including oral or inhaled corticosteroids and/or β2 agonists. None of the asthmatic or COPD patients experienced a recent (<3 mo) exacerbation of the disease. The mean duration of the disease in asthmatic and COPD patients was 26±4.6 and 18±2.8 yr, respectively. Of the 19 control subjects who received no treatment, 8 of them were lifelong nonsmokers, whereas 11 were former smokers.

Asthmatic patients were enrolled using the following inclusion criteria. Patients >18 yr had to exhibit characteristic symptoms (i.e. wheezing and breathlessness), as well as bronchial hyper-responsiveness confirmed either by a significant improvement by >15% in the forced expiratory volume in 1 s (FEV1) 10 min after the inhalation of 200 μg of salbutamol, or a provocative concentration of methacholine required to lower the FEV1 by 20% (PC20) of <4 mg/ml according to the American Thoracic Society criteria. Patients must all be graded as severe persistent asthmatics according to the Global Initiative for Asthma guidelines based on both clinical features and daily medication regimen. All patients must also be lifelong nonsmokers with no recent exacerbation (<3 mo). Exclusion criteria were recent bacterial or viral infections (<3 mo).

COPD patients were enrolled using the following inclusion criteria. Patients >18 yr had to present chronic symptoms (i.e. cough and breathlessness) and poorly reversible airflow obstruction defined by both an improvement of <10% in the FEV1 10 min after the inhalation of 200 μg of salbutamol or ipratropium bromide, and a postbronchodilator FEV1/forced vital capacity ratio <70% according to the Global Initiative for Chronic Obstructive Lung Disease (GOLD) guidelines. Patients must be graded as moderate to severe COPD according to the GOLD guidelines. Exclusion criteria were recent bacterial or viral infections (<3 mo).

Control subjects were enrolled using the following inclusion criteria. Subjects >18 yrs had to have normal lung function test and have underwent a fiberoptic fibroscopy or a thoracotomy because of hemoptysis or abnormal image on x ray. They must be asymptomatic without any treatment, and only those in whom fiberoptic investigation and bronchial mucosa were normal were selected as controls. Exclusion criteria were any co-morbidity, such as asthma or COPD, and recent bacterial or viral infections (<3 mo).

The study followed recommendations outlined in the Helsinki Declaration and received approval from the local ethics committee.

TABLE 1
Clinical And Functional Characteristics of Subjects

| | Patients | | |
|---|---|---|---|
| Characteristic | with asthma | Patients with COPD | Controls |
| No. of patients | 14 | 17 | 19 |
| Age (yr) | 42.3 ± 5.7 | 59.8 ± 3.0 | 60.4 ± 2.5 |
| BMI (kg/m²) | 26.9 ± 1.1 | 24.0 ± 0.9 | 27.4 ± 1.1 |
| Smoking history | | | |
| Pack years | 0 | 46.3 ± 4.8 | 19.5 ± 5.7 |
| Current (no. of | 0 | 8 | 0 |
| patients) | | | |
| Former (no. of | 0 | 9 | 11 |
| patients) | | | |
| Years since quitting | — | 11.4 ± 4.8 | 13.1 ± 3.8 |
| Treatments | | | |
| LABA (no. of | 11 | 6 | 0 |
| patients) | | | |
| ICS (no. of patients) | 10 | 5 | 0 |
| OCS (no. of patients) | 6 | 3 | 0 |
| FEV₁ | | | |
| Liters | 2.2 ± 0.2 | 1.8 ± 0.1 | 2.9 ± 0.1 |
| Percentage of | 83.1 ± 6.3 | 60.2 ± 2.5 | 101.7 ± 3.2 |
| predicted value | | | |
| FEV₁:FVC ratio (% of | 71.9 ± 3.7 | 56.3 ± 2.2 | 81.8 ± 1.7 |
| FVC) | | | |
| FEF 25-75 | | | |
| Liters sec⁻¹ | 1.9 ± 0.3 | 0.9 ± 0.1 | 2.9 ± 0.1 |
| Percentage of | 54.6 ± 7.8 | 28.6 ± 3.1 | 90.2 ± 4.0 |
| predicted value | | | |

Data are the mean ± the SEM.
BMI, body mass index;
LABA, long-acting β2 agonist;
ICS, inhaled corticosteroid;
OCS, oral corticosteroid;
FEV₁, forced expiratory volume in one second;
FVC, forced vital capacity;
FEF 25-75, forced expiratory flow between 25 and 75% of FVC.

### EXAMPLE 2

### Experimental Procedures

### Bronchial Specimens

Bronchial specimens were obtained by either fiberoptic bronchoscopy or lobectomy, as previously described (Berger et al., FASEB J. 17:2139-2141 (2003); El-Shazly et al., J. Immunol. 176:1860-1868 (2006)). Fiberoptic bronchoscopy was performed after the anesthesia of the upper airways was achieved with lidocaine 10% spray. The fiberoptic bronchoscope (FB-5V; Pentax) was introduced, and biopsies were taken from various bronchial carinae from the middle lobe.

Because the smooth muscle remodeling in both asthma and COPD occurred at two different bronchial localizations, asthmatic bronchial specimens were thus collected from the third to the fourth generation, those from COPD patients from the fourth to the sixth generation and those from control subjects were collected from the third to the sixth generation. Specimens were immediately transferred to the laboratory in a sterile container containing DME.

### Optic Microscopy and Immunohistochemistry

Bronchial specimens were fixed in 10% buffered formalin and processed in standard fashion for paraffin embedding. Three μm-thick sections were cut and stained with hematoxylin-eosin-safran or processed for immunohistochemistry. After deparaffinization and rehydratation through graded alcohols, endogenous peroxidase was inhibited using a solution of 0.1% sodium azide and 0.3% hydrogen peroxide for 30 min, followed by two rinses in PBS (Invitrogen). Nonspecific staining was blocked using 10% BSA (Dako) for 30 min. Sections were incubated for 2 h at room temperature with mouse anti-human smooth muscle actin (clone 1A4) or an unrelated mouse antibody (both from Sigma-Aldrich). After rinsing in PBS, biotinylated rabbit anti-mouse F(ab′)2 (Dako) was applied to the sections for 2 hours, followed by the streptavidin-biotinylated horseradish peroxidase complex (Dako) for an additional 2 hours. After rinsing in PBS, 1 mg/ml 3,3′-diaminobenzidine tetrahydrochloride (Dako), plus 0.02% hydrogen peroxide for 6 min, were used as substrate to develop a peroxide-dependent brown color reaction at room temperature. Finally, the sections were rinsed and counterstained with Mayer's hematoxylin. There was assessable BSM in the bronchial specimens from all 14 asthmatics, 17 COPD patients, and 19 controls. The total area of smooth muscle layer was assessed manually in a blinded fashion using ScanView software (Soft Imaging System) at a magnification of 200×. This smooth muscle area was normalized by the whole area of the corresponding tissues and presented as percentages of whole area.

### Electron Microscopy

Specimens were fixed in 2.5% glutaraldehyde in cacodylate buffer, postfixed in 1% osmium tetroxide, dehydrated, and embedded in epon. For each specimen, semithin sections (1 μm thick) were cut and stained with alkaline toluidine blue. The first semithin section large enough to span from the epithelium to the muscular layer was selected. Ten ultrathin serial sections (60 nm thick) were then cut on diamond knives. Three of these latter sections were subsequently randomly selected and placed on grids. Staining was performed with uracile acetate and lead citrate. Grids were then scanned by transmission electron microscopy (Tecnai 12; Philips) and examined by a pathologist from left to right and from top to bottom to locate every whole nucleated BSM, epithelial, and endothelial cell. Each ultrathin section was examined in its entirety. To evaluate the number of mitochondria, computerized photographs and measurements were performed in a blinded fashion by using ScanView software at a standard magnification of 6,000×. Mitochondrial densities were performed using Image J 1.34s free software (National Institutes of Health).

### Cell Cultures

Primary cultures of BSM cells were established from bronchial specimens. After a fine dissection under a microscope, smooth muscle explants were cultured in six-well plates in a humidified atmosphere at 37° C. with 5% CO2. Cells were maintained in sterile DME containing 10% (vol/vol) FCS and 4.5 g/liter glucose, supplemented with 2 mM 1-glutamine, 100 U/ml penicillin, 100 mg/ml streptomycin, and 0.25 mg/ml amphotericine B (antimycotic/antibiotic solution; all from Invitrogen), 1 mM sodium pyruvate, and 1% (vol/vol) nonessential amino acid mixture (both from Sigma-Aldrich). The medium was changed every 48-72 h. After 6-8 wk, confluent cells were rinsed twice with HBSS and passaged with trypsin-EDTA (both from Invitrogen).

To assess purity of the cells, an immunocytochemical method was used, using an indirect immunofluorescence technique. Cells of varying passage number were growth arrested by incubating the cells with serum-free DME supplemented with 10 μg/ml insulin, 5.5 μg/ml transferrin, 5 ng/ml selenium, 0.5 μg/ml BSA, and 4.7 μg/ml linoleic and oleic acid (ITS solution; Sigma-Aldrich). After 48 h, cells were rinsed twice in PBS and fixed with cold methanol for 20 min. Nonspecific staining was blocked using PBS containing 3% BSA for 30 min. Monoclonal antibodies (mAb) diluted in PBS with 1% BSA, including anti-α-smooth muscle actin (Sigma-Aldrich), anti-smooth muscle myosin (Sigma-Aldrich), anti-cytokeratin 18 (Sigma-Aldrich), and anti-factor VIII (Dako) were incubated for 1 h. Control slides were treated similarly, using an unrelated antibody (mouse IgG; Sigma-Aldrich). After rinsing with PBS containing 0.05% Tween20 (Sigma-Aldrich), the cells were incubated for 1 h with FITC-conjugated anti-mouse immunoglobulins (Dako). Slides were mounted with a drop of fluorescent mounting medium (Dako) and observed under a fluorescence microscope (Nikon). Depending on the experiments, cells with a confirmed smooth muscle phenotype were seeded on coverslips, chamber slides, 75-cm² culture flasks, and 6-, 12-, or 96-well plates. Only cells at passage 2 to 4 were used for this study.

To analyze the mitochondrial ultrastructure, BSM cells were growth arrested, as described in the previous section, for 6 d. Three million cells were then collected and centrifuged at 400 g for 5 min. Cell pellets were fixed in 2.5% glutaraldehyde for 2 h at 4° C. and processed for electron microscopy, as described in the previous section. Growth curves were built to assess the role of mitochondria in cell proliferation. For this purpose, 25,000 BSM cells were plated on 75-cm² flasks and cultured with 10% FCS-DME containing either 4.5 g/liter galactose or 4.5 g/liter glucose. In another set of experiments, we investigated the effects of 50 μg/liter ethidium bromide (Qbiogene) for up to 33 d, 1.3 g/liter cyclic cGMP (3 mM; Sigma-Aldrich) for up to 6 d, or 0.5 g/liter D600, i.e., methoxyverapamil or gallopamil (1 μM; Sigma-Aldrich) for up to 2 d in 10% FCS-DME containing 4.5 g/liter glucose. For these latter experiments, medium was changed every 24 h, and, at the indicated time, BSM cells were harvested by trypsinization and counted in triplicate using a cytometer.

### Immunoblotting

Whole lysates from BSM cells were collected using 1% Triton X-100 lysis buffer in the presence of 2 mM Na orthovanadate, 1 mM EDTA, 50 μg/ml aprotinin, 100 μM leupeptin, 1 mM 1.4 Dithio-DL-Treitol, and 1 mM amino-ethyl-benzenesulfonyl fluoride hydrochloride (all from Sigma-Aldrich). Cellular extracts were reduced with mercaptoethanol, subjected to electrophoresis on a 10% acrylamide reducing gel, and transferred to PVDF membranes (Immobilon TM-P; Millipore). The immunoblots were then developed using mouse anti-human porin (MitoSciences), mouse anti-human α-actin (Sigma-Aldrich), rabbit anti-human phospho-CaMK-IV, rabbit anti-human PGC-1α, rabbit anti-human NRF-1, or rabbit anti-human mitochondrial transcription factor A (mtTFA; all purchased from Santa Cruz Biotechnology, Inc.) for 14 h at 4° C. For amplification, biotinylated goat secondary antibody anti-mouse IgG (Bio-Rad Laboratories) or anti-rabbit IgG (Santa Cruz Biotechnology, Inc.) for 2 h at room temperature and a streptavidin-biotinylated horseradish peroxidase complex (Dako) were used. Immunoblots were revealed by enhanced chemiluminescence (Uptima). Blot images were acquired using BioCaptMW (Thermo Fisher Scientific), and band densities were quantified using ImageJ software.

### Endogenous Cell Oxygen Consumption and Determination of the Coupling Ratio

Endogenous cellular oxygen consumption was monitored on intact cells at 37° C. in a 1-ml thermostatically controlled chamber (106 cells/ml/run) equipped with a Clark oxygen electrode (Oxygraph System; Hansatech). The respiratory buffer was the glucose or the galactose culture medium described in Cell culture. The endogenous respiratory rates were expressed in ngatom 0/min/10⁶ cells. To assess the mitochondrial coupling, respiratory rates were monitored using inhibitors of the phosphorylation system, i.e., 1 mM atractyloside, 1 mM carboxyatractyloside, and 1 nM oligomycin in combination. Such inhibitors are considered as decouplers and can be used to evaluate the degree of mitochondrial ATP synthesis. Control experiments were performed using 1 mM cyanide as a blocker of the respiratory chain.

### Three-Dimensional Assessment of the Mitochondrial Network Organization

To visualize the mitochondrial network, GFP was targeted to the mitochondrial matrix space of the BSM cells. For this purpose, the mitochondrion-targeted GFP plasmid (mito-GFP), which was derived from the plasmid pEGFP-N1 (Clontech Laboratories, Inc.) containing the leader sequence of the E1α subunit of pyruvate dehydrogenase was used. One day before transfection, BSM cells were plated at 50% confluency in 10% FCS-DME without antibiotics. Transfections were conducted for 6 h at 37° C. in 5% CO2 with 1.5 μg of plasmid and 1.7% of the cationic lipid Lipofectamine 2000 (Invitrogen) in the absence of FCS. After 72 h, BSM cells were rinsed in PBS and fixed with 4% paraformaldehyde (PFA) for 20 min on ice. After drying, the slides were mounted with fluorescent mounting medium (Dako). Confocal differential interference contrast images were obtained using the FluoView laser scanning microscope (Nikon) and 60× oil-immersion objective. Z series sections were recorded in successive z axis serial sections at 0.5-μm intervals, and they were composed of optical sections in the x-y optical plane. Sections were reconstituted in three-dimensional images using Imaris Software (Bitplane).

### RNA Extraction, RT, and Real-Time Quantitative PCR

BSM cells were washed twice with HBSS and lysed by the addition of 300 liters/well Trizol (Invitrogen) and 30 μl of chloroform (Sigma-Aldrich). The RNA was extracted from the aqueous phase after being centrifuged at 12,000 g for 15 min. RNA was precipitated in the presence of isopropanol (Sigma-Aldrich) at 220° C. overnight. The pure RNA was recovered by centrifugation and washed with 80% ethanol (Sigma-Aldrich). The purity was assessed by electrophoresis (30 mA for 75 min) on 1.5% agarose gel (Bio-Rad Laboratories), followed by 20 min incubation in 10 μg/ml ethidium bromide (Sigma-Aldrich), showing 2 bands corresponding to the 28S and 18S fractions of ribosomal RNA. The concentration of RNA was measured spectrophotometrically by GeneQuant RNA/DNA calculator (GE Healthcare). The total RNA (1 μg) was reverse transcribed into cDNA using AMV RT (Promega), RNase inhibitor, and oligo d(T) as a primer at 42° C. for 60 min, followed by heating at 94° C. for 3 min.

Real-time quantitative PCR was performed on a Rotor-Gene 2000 (Corbett Research). In brief, triplicate PCR reactions were assembled in 0.1-ml strip tubes containing cDNA from 10 ng of total RNA, 0.2 μl 50× Titanium Taq DNA Polymerase, 1× Titanium Taq PCR Buffer (Clontech Laboratories, Inc.), 1 mM dNTP, 100 mM each of the appropriate primer, and 0.5× Sybr Green I (Invitrogen). The PCR was performed under the following conditions: denaturation at 95° C. for 15 s, annealing temperature (64-72° C.) depending on specific primers for 15 s, and extension at 70° C. for 30 s for 30-40 cycles. Data collection was performed after each extension step, at a temperature at least 3.5° C. lower than the melting temperature of the amplicon (generally between 80-85° C.) to eliminate nonspecific fluorescence signal. PCR-negative controls were systematically made using water instead of cDNA or RNA sample without the RT step. All specific primers were designed using the primer analysis software (Oligo 6.6; Molecular Biology Insights) and were ordered from Sigma-Aldrich. Primers, sense and antisense, were as follows: for mtTFA (NM_{—}003201), forward 5′-GAAGTCGACTGCGCTCCC-3′ (SEQ ID NO:1) and reverse 5′-ACTCCGCCCTATAAGCATC-3′(SEQ ID NO:2); for PGC-1α( (NM_{—}013261), forward 5′-GTCACCACCCAAATCCTTATT-3′(SEQ ID NO: 3) and reverse 5′-GGCGATCTTGAACGTGAT-3′ (SEQ ID NO: 4); for NRF-1 (NM_{—}005011), forward 5′-AAGATCAGCAAACGCAAACAC-3′ (SEQ ID NO: 5) and reverse 5′-CCCGTACCAACCTGGATAAGT-3′ (SEQ ID NO: 6); for tyrosin 3-monooxygenase/tryptophan 5-monooxygenase activation protein, zeta polypeptide (YWHAZ) (NM_{—}003406), forward 5′-GGGGATGTGGAATTTTTATACAAG-3′ (SEQ ID NO: 7) and reverse 5′-CGCCAGGACAAACCAGTATGTAG-3′ (SEQ ID NO: 8); for hypoxanthine phosphoribosyltransferase 1 (HPRT-1) (NM_{—}000194), forward 5′-GTGAAAAGGACCCCACGAA-3′ (SEQ ID NO: 9) and reverse 5′-AGTCAAGGGCATATCCTACAACAA-3′ (SEQ ID NO: 10) ; and for human acidic ribosomal phosphoproteins (PO) (NM_{—}001002), forward 5′-CAACGGGTACAAACGAGTC-3′ (SEQ ID NO: 11) and reverse 5′-CTTCCTTGGCTTCAACCTTAG-3′ (SEQ ID NO: 12). The efficiency of the PCR reactions was always >90%. The specificity of the amplified PCR products was examined with the melting curve analysis, and also in 2% agarose gel containing ethidium bromide. The RT-PCR expression of the target genes (i.e., mtTFA, PGC-1α, and NRF-1) was thus presented as an arbitrary unit and normalized to endogenous references (geometric averaging of three internal control genes; i.e., YWHAZ, HPRT-1, and PO) according to geNorm software.

### Microspectrofluorimetry

Change in growth-arrested BSM cell intracellular calcium concentration ([Ca²⁺]ᵢ), was assessed using the Ca²⁺-sensitive probe indo-1, as previously described (Berger et al., J. Appl. Physiol. 91:995-1003 (2001); Trian et al., Am. J. Respir. Cell Mol. Biol. 34:49-55 (2006)). In brief, cells were loaded with indo-1 (Calbiochem) and mounted in a perfusion chamber continuously perfused with phosphate salt solution containing either 2 mM Ca2+ in the absence or presence of 1 μM D600, or 400 μM EGTA (Sigma-Aldrich) in the absence of extracellular calcium. BSM cells were stimulated with 10⁻⁵ M acetylcholine or histamine (both from Sigma-Aldrich). Individual cell calcium levels were monitored continuously. Results were expressed as the mean±the SEM of resting or peak of [Ca²⁺]ᵢ rises (in millimoles) and area under the curves (nanomoles/second). Experiments were carried out at room temperature (22-25° C.).

### BrdU Incorporation

Growth-arrested BSM cells were cultured in 96-well plates for 24 h and stimulated with 10% FCS-DME containing glucose, galactose, ethidium bromide, or cyclic GMP for 24 h or containing D600 for 48 h, as described above. Control BSM cells remained in serum-deprived DME. DNA synthesis was measured using the BrdU kit according to the manufacturer's instructions (Roche). In brief, BSM cells were incubated with 10 μM BrdU for 2 h at 37° C. and fixed for 20 min at room temperature. Cells were denaturized for 30 min and incubated with the anti-BrdU antibody for 2 h at room temperature. Absorbance was measured at 370 nm in a microplate reader in triplicate. Results are expressed as a normalized ratio of BrdU incorporation, i.e. absorbance for test condition divided by absorbance for control serum-deprived condition.

### Statistical Analysis

The statistical analysis was performed with NCSS 2001 software. Comparisons between the three groups were performed by means of ANOVA, with the use of Bonferroni's test for multiple comparisons or paired Student's t tests. Values are presented as the mean±the SEM. A Pearson correlation matrix was built between in vitro and in vivo measurements. A P value <0.05 was considered statistically significant.

### EXAMPLE 3

### Mitochondrial Mass and Activity are Increased Only in the BSM of Asthmatics

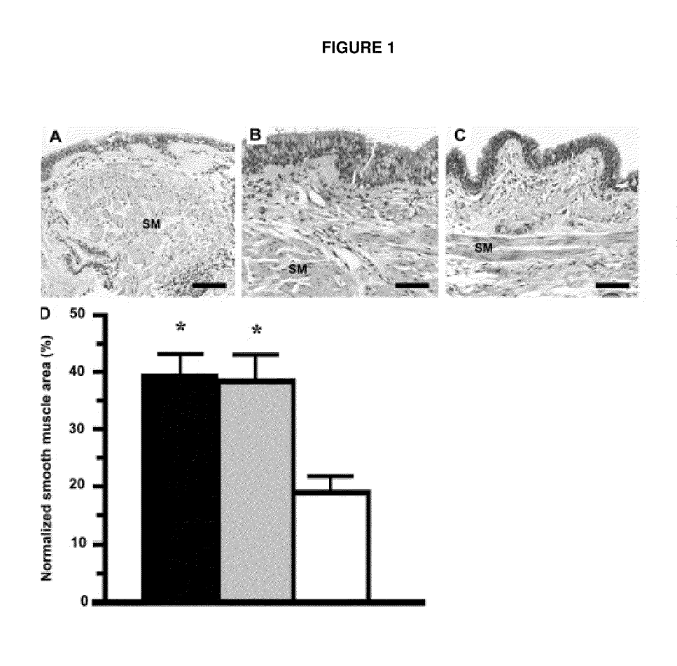

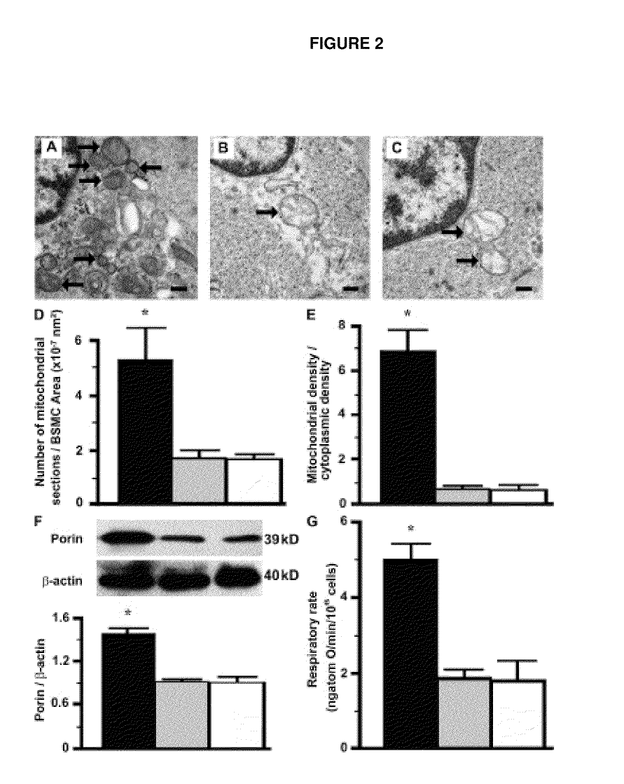

A morphological analysis of BSM was performed in the three groups of subjects (FIG. 1, A-C). BSM mass was increased in both asthmatic and COPD patients as compared with controls (FIG. 1D; P=0.01). Ultrastructure of BSM mitochondria was then compared in asthma and COPD to control subjects (FIG. 2, A-C). The number of mitochondrial sections was higher in the BSM of asthmatics than in that of both COPD and controls (FIG. 2D; P<0.001). The mean area per section was, however, unchanged (0.1±0.007, 0.1±0.016, and 0.1±0.009 μm² for asthmatics, COPD, and controls, respectively; analysis of variance [ANOVA] P=0.87). Collectively, these results favor the hypothesis that the increase in mitochondrial mass in the BSM of asthmatics is related to an increase in number rather than in individual size. Similar results were obtained with cultured growth-arrested BSM cells.

There was a significant increase in the mitochondrial density in asthmatic BSM cells both ex vivo (FIG. 2E; P=0.01) and in vitro (data not shown) as compared with both COPD and controls. Based on electronic microscopy, the mitochondrial density has been shown to reflect the mitochondrial activity (13, 14). To assess the smooth muscle specificity of these results, ultrastructural mitochondrial parameters in other cell types from the same bronchial specimens were also analyzed. There was no difference between the three groups, in terms of both number (P=0.35 and P=0.89) and density of mitochondria (P=0.47 and P=0.87) in endothelial and epithelial cells, respectively (unpublished data). The increased mass of asthmatic BSM mitochondria was further confirmed, in vitro, by an increase in the porin content compared with that of both COPD and controls (FIG. 2F; P=0.001).

It is also found within the asthmatic population that both duration of the disease and forced expiratory volume in one second (FEV₁)/forced vital capacity (FVC) ratio were correlated with the porin content (r=0.77, P=0.03 and r=−0.84, P=0.01, respectively), whereas no correlation was found within the COPD or the control population. Similarly, the oxygen consumption of asthmatic BSM cells was specifically enhanced compared with COPD or control BSM cells (FIG. 2G; P=0.01). Such an increase in the mitochondrial respiratory rate was not associated with differences in the coupling degree, as demonstrated by the effects of inhibitors of the phosphorylation system. Similarly, oxygen consumption of COPD and control BSM, although lower, also reflected coupled mitochondrial respiration (data not shown). These inhibitors decreased the mitochondrial respiration of asthmatics, COPD, and controls by 18.6, 21.2, and 24.7%, respectively, whereas cyanide completely inhibited oxygen consumption of all BSM cells. The increased mitochondrial respiration in asthma thus appears to result from the increased organelle content and subsequent enhancement in the overall oxidative capacity in BSM.

### EXAMPLE 4

### Mitochondrial Biogenesis is Increased in Asthmatic BSM Cells Through a Calcium-Dependent Pathway

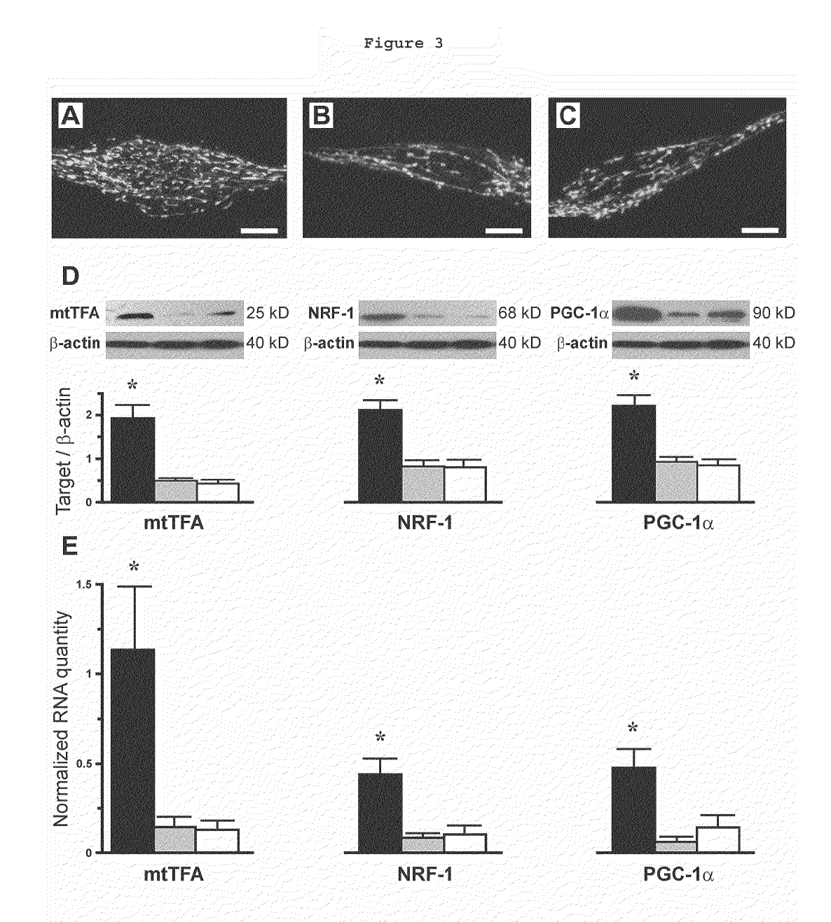

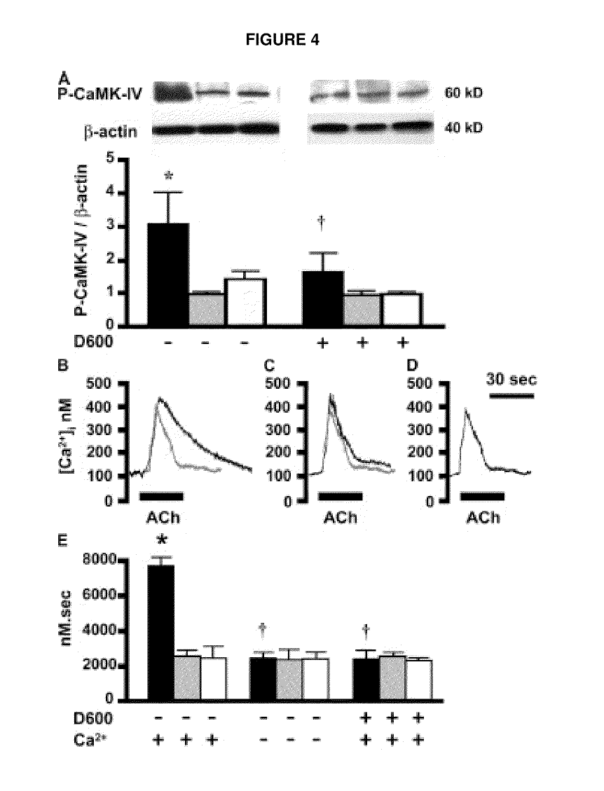

Next, the mitochondrial network was analyzed by confocal microscopy (FIG. 3, A-C). Surprisingly, asthmatic BSM cells presented a typical aspect of intense mitochondrial biogenesis, as shown by the presence of several budding areas with intense dots, and a wider network with increased ramification (FIG. 3A). Because the mitochondrial transcription factor A (mtTFA) is the main factor involved in mitochondrial biogenesis, protein content and transcription level in the three groups of BSM were measured. As compared with both controls and COPD, mtTFA was increased in asthmatic BSM cells (FIG. 3, D and E). Upstream mechanisms that would explain such an increased mitochondrial biogenesis were then examined. The two transcription factors nuclear respiratory factor-1 (NRF-1) and peroxisome proliferator-activated receptor α coactivator-1α (PGC-1α) were both elevated in asthmatic BSM cells at both the protein and the transcription levels (FIG. 3, D and E). Among the various factors that could activate PGC-1α, it was observed that the calcium/calmodulin-dependent protein kinase IV (CaMK-IV) was phosphorylated and thus activated in asthmatic BSM cells compared with controls and COPD (FIG. 4A).

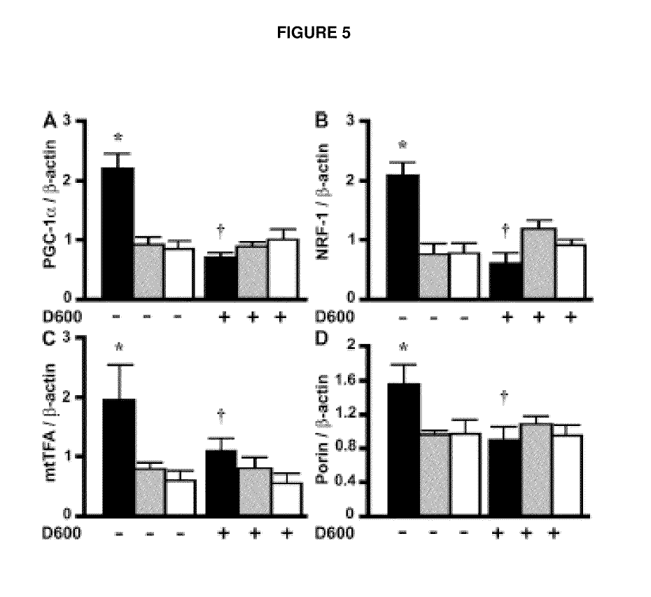

Next, whether calcium homeostasis was deregulated in asthmatic BSM cells was investigated using micro-spectrofluorimetry. The resting calcium concentration was consistent in asthmatic (125±8 nM), COPD (127±7 nM), or control BSM cells (133±4 nM; P=0.60) However, the calcium response to acetylcholine was significantly altered in asthmatic BSM cells (FIG. 4, B-D). Whereas the amplitude of the calcium rise was unchanged (275±20, 284±43, and 272±27 nM in asthmatics, COPD, and controls, respectively; P=0.95), the area under the curve was significantly increased in asthmatic BSM cells (FIG. 4E). A similar result was obtained in asthmatic BSM cells stimulated with histamine, confirming that it was not agonist-specific (data not shown). An enhanced calcium influx in asthmatic BSM cells accounts for this result, as both removal of extracellular calcium using EGTA and blockade of calcium channels using methoxyverapamil (gallopamil or D600) abolished such abnormal calcium responses in asthmatic BSM cells (FIG. 4E data not shown). Blockade of calcium influx using D600 also inhibited the activation of CaMK-IV (FIG. 4A) and the subsequent activation of mitochondrial biogenesis through PGC-α, NRF-1, and mtTFA (FIG. 5, A-C), leading to the increase in the mitochondrial mass as assessed by the porin content (FIG. 5D). Thus, these results indicate that an enhanced extracellular calcium influx, specific to asthmatic BSM cells, is the initial priming event leading to an increased mitochondrial biogenesis and mass.

### EXAMPLE 5

### Only Asthmatic BSM Cell Proliferation is Mitochondria Dependent

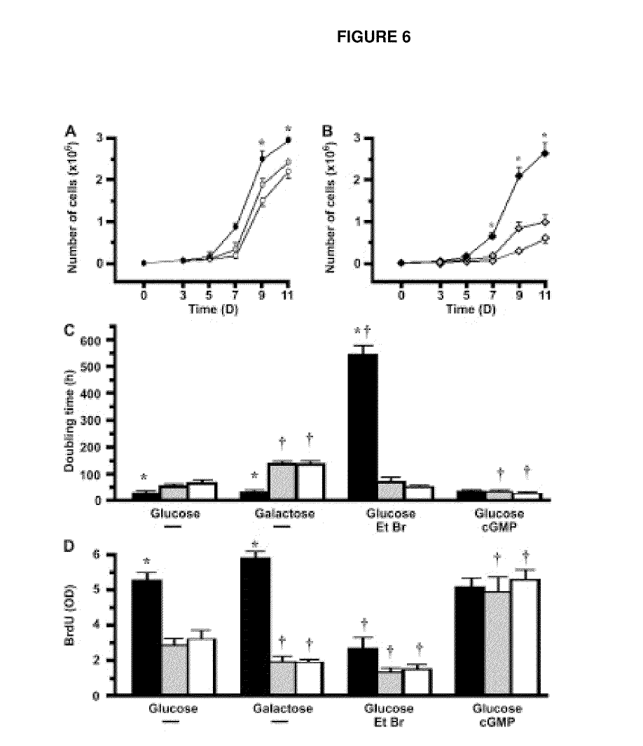

To further assess the specific role of mitochondria in asthmatic BSM proliferation contributing to airway remodeling, BSM cell growth in the presence or absence of anaerobic glycolysis was compared. For this purpose, BSM cell proliferation curves were plotted in the three groups of subjects using either glucose or galactose in the culture medium (FIG. 6, A and B). In the presence of glucose, which allows ATP to be produced by both aerobic and anaerobic glycolysis, asthmatic BSM cell growth was significantly increased compared with that of COPD and control subjects (FIG. 6A; P=0.02), with a concomitant decrease in the doubling time (FIG. 6C; P=0.02). When galactose, which only allows cells to produce ATP by mitochondrial oxidative phosphorylations, was substituted for glucose, the doubling time of the cell growth from both COPD (P=0.02) and control subjects (P=0.02) significantly increased, whereas that of asthmatic cells remained constant (FIG. 6, B and C).

Because an increase in cell growth can be related to a decreased apoptosis and/or an increased proliferation, Annexin V binding and BrdU incorporation were analyzed, respectively. Taking into account the percentage of Annexin V-positive cells, spontaneous apoptosis was not altered in asthmatic BSM cells as compared with that of COPD and controls (data not shown). However, BrdU incorporation increased in BSM cells from asthmatics as compared with that in both COPD and controls in the presence of glucose (FIG. 6D; P=0.01). Incubation for 11 d in the absence of glucose significantly inhibited BrdU incorporation in BSM cells from controls (P=0.02) and COPD patients (P=0.02), but not in that from asthmatics (FIG. 6D; P=0.99). Collectively, these results demonstrate that the proliferation of asthmatic BSM cells mainly uses mitochondrial-dependent oxidative phosphorylations, whereas that of COPD and control subjects mainly uses mitochondrial-independent anaerobic glycolysis.

### EXAMPLE 6

### Increased Mitochondrial Mass Explains Increased Asthmatic BSM Cell Proliferation

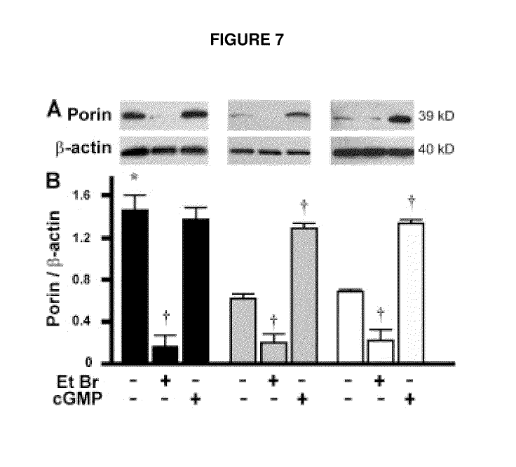

To determine whether the increased mitochondrial biogenesis found in asthmatic BSM cells is a cause or a consequence of the asthmatic BSM cell increased proliferation, three alternative approaches were used. First, mitochondria-deficient BSM cells were generated using culture with ethidium bromide, as previously described (10, 15). After 16 d of culture, all of the asthmatic BSM cells died, whereas those from both COPD patients and controls were still alive up to 30 d in the ethidium bromide medium (unpublished data). 6 d of incubation with ethidium bromide significantly decreased the amount of porin in the three groups of subjects (FIG. 7). FIG. 6C demonstrates that ethidium bromide significantly increased the doubling time of asthmatic BSM cells (P<0.001), but did not change that of both COPD (P=0.15) and control cells (P=0.09). Similarly, proliferation of mitochondria-deficient asthmatic BSM cells decreased (P<0.001), whereas that of COPD and controls remained unchanged (data not shown). However, ethidium bromide also decreased BrdU incorporation of BSM cells from the three groups of subjects (FIG. 6D).

In a second alternative approach, mitochondrial biogenesis was stimulated using cyclic GMP for 6 d as previously described (16). The amount of mitochondria increased in such stimulated BSM cells from both COPD and controls (FIG. 7; P=0.02 for both). Because the amount of mitochondria was already up-regulated in BSM cells from asthmatics, cyclic GMP failed to additionally increase the porin content (P=0.2). Cyclic GMP significantly decreased the doubling time (P=0.01), and increased the BrdU incorporation (P=0.02) and the proliferation of BSM cells from COPD patients (P=0.01) (FIG. 6, C and D). Similarly, cyclic GMP also decreased the doubling time (P<0.001) and increased the BrdU incorporation (P<0.001) and the proliferation of BSM cells from controls (P<0.001; FIG. 6, C and D, and data not shown). However, cyclic GMP had no effect in asthmatic BSM cell growth and proliferation.

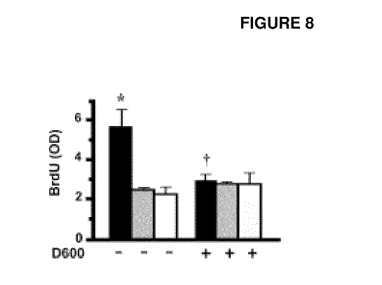

Finally, in a last approach, the effect of altering calcium homeostasis on the proliferation of BSM cells was analyzed. D600, which blocks calcium influx (FIG. 4E) and the resulting increase in mitochondrial biogenesis and content only in asthmatic BSM cells (FIG. 5), also significantly inhibited the increased BrdU incorporation in BSM cells from asthmatics, thus confirming that this enhanced cellular calcium influx represents the initial priming event (FIG. 8).

### EXAMPLE 7

### Calcium Channel Inhibitors Decreases Asthmatic Smooth Muscle Remodeling In Vivo

The effects of calcium channel inhibitors such as gallopamil on asthmatic smooth muscle remodeling can be examined with severe asthmatic or non severe asthmatic patients. A phase 2 randomized double blind study against placebo is conducted. Since inflammation also activates mitochondrial biogenesis in BSM cells, optimization of asthma treatment is first effected during 3 months by both controlling co morbidities and decreasing bronchial inflammation using exhaled NO and eosinophil count within the induced sputum. Patients are subject to fiberoptic fibroscopies before and after 12-month treatment with gallopamil/placebo. Same proceedings are conducted with calcium channel inhibitors, such as verapamil, devapamil, emopamil, anipamil, nifedipine, nicardipine, or diltiazem. Also, treatments are performed in combination with standard care therapy for asthma, such as (1) inhaled corticosteroids such as beclomethasone (up to 2000 μg/day equivalent), or budesonide, or fluticasone, and (2) LABA (Long acting beta 2 agonists), such as formoterol or salmeterol, and (3) optionally a third one which is an oral corticoid such as prednisolone.

Inclusion criteria include male or female aged more than 18 yrs; written informed consent; and a diagnosis of severe asthma according to ATS criteria. Main exclusion criteria include smoker or former smoker; chronic viral infections (hepatitis, HIV); pregnancy or breastfeeding; contraindications to gallopamil or bronchoscopy.

The primary outcomes are bronchial smooth muscle remodeling assessed by the smooth muscle size within the bronchial mucosa, the smooth muscle cell size, the number of smooth muscle cells/area of smooth muscle, and the proliferation rate of the smooth muscle cells. Secondary outcomes include: bronchial thickness assessed by 3D analysis of computed tomography; asthma control using the asthma control questionnaire (ACQ) validated by Juniper; and BSM mitochondrial number and activity assessed by electronic microscopy, oxygraphy, or Western blot.

The clinical study comprises 3 phases. The initial phase (3 months) includes initial clinical and functional assessment of asthma control including ACQ, plethysmography, exhaled NO, and induced sputum; Co-morbidity treatment; and optimization of asthma treatment. After the first fiberoptic fibroscopy, the treatment phase lasts 12 months, and the final phase (3 months) includes fiberoptic fibroscopy and 3 month follow-up after stopping the treatment.

### REFERENCES

1. Busse and Lemanske Jr. 2001. N. Engl. J. Med. 344:350-362.
2. Pepe et al. 2005. J. Allergy Clin. Immunol. 116:544-549.
3. Hogg et al. 2004. N. Engl. J. Med. 350:2645-2653.
4. Lambert et al. 1993. J. Appl. Physiol. 74:2771-2781.
5. Johnson et al. 2001. Am. J. Respir. Crit. Care Med. 164:474-477.
6. Roth et al. 2004. N. Engl. J. Med. 351:560-574.
7. Ramos-Barbon et al. 2005. J. Clin. Invest. 115:1580-1589.
8. Jeffery. 2001. Am. J. Respir. Crit. Care Med. 164:S28-S38.
9. Springer et al. 2004. Biol. Chem. 385:649-653.
10. Holmuhamedov et al. 2002. FASEB J. 16:1010-1016.
11. Green and Kroemer. 2004. Science 305:626-629.
12. Oltersdorf et al. 2005. Nature 435:677-681.
13. Hackenbrock et al. 1971. J. Cell Biol. 51:123-137.
14. Rossignol et al. 2004. Cancer Res. 64:985-993.
15. King and Attardi. 1989. Science 246:500-503.
16. Nisoli et al. 2004. Proc. Natl. Acad. Sci. USA. 101:16507-16512.
17. Ebina et al. 1993. Am. Rev. Respir. Dis. 148:720-726.
18. Bai et al. 2000. Am. J. Respir. Crit. Care Med. 162:663-669.
19. Benard et al. 2006. Am. J. Physiol. Cell Physiol. 291 :C1172-C1182.
20. Rasmussen et al. 2003. Exp. Gerontol. 38:877-886.
21. Hansson et al. 2004. Proc. Natl. Acad. Sci. USA. 101:3136-3141.
22. Lehman et al. 2000. J. Clin. Invest. 106:847-856.
23. Wu et al. 1999. Cell 98:115-124.
24. Gleyzer et al. 2005. Mol. Cell. Biol. 25:1354-1366.
25. Nisoli et al. 2003. Science 299:896-899.
26. Pilz and Casteel. 2003. Circ. Res. 93:1034-1046.
27. Miura et al. 2006. Am. J. Pathol. 169:1129-1139.
28. Parenti et al. 1998. J. Biol. Chem. 273:4220-4226.
29. Wu et al. 2002. Science 296:349-352.
30. Sato et al. 2006. Nat. Med. 12:1410-1416.
31. Bootman and Berridge. 1995. Cell 83:675-678.
32. Massey et al. 1988. J. Allergy Clin. Immunol. 81:912-918.
33. Ahmed et al. 1988. J. Allergy Clin. Immunol. 81:852-858.
34. Hood. 2001. J. Appl. Physiol. 90:1137-1157.
35. Global Initiative for Asthma. 1995. Global strategy for asthma management and prevention. NIH publication (updated 2005).
36. Global Initiative for Chronic Obstructive Lung Disease. 1998. Global strategy for the diagnosis, management and prevention of chronic obstructive pulmonary disease. NIH publication (updated 2005).
37. Berger et al. 2003. FASEB J. 17:2139-2141.
38. El-Shazly et al. 2006. J. Immunol. 176:1860-1868.
39. Begueret et al. 2007. Thorax 62:8-15.
40. Trian et al. 2006. Am. J. Respir. Cell Mol. Biol. 34:49-55.
41. Berger et al. 2001. J. Appl. Physiol. 91:995-1003.

What is claimed is:

1. A method of inhibiting bronchial smooth muscle remodeling in asthma, comprising the step of administering to a subject having asthma a therapeutically effective amount of an agent that inhibits calcium-dependent mitochondrial biogenesis.

2. The method of claim 1, wherein the agent inhibits calcium influx in bronchial smooth muscle cells or inhibits proliferation of bronchial smooth muscle cells.

3. The method of claim 1, wherein the agent is a calcium channel blocker.

4. The method of claim 3, wherein the agent is selected from the group consisting of gallopamil verapamil, devapamil, emopamil, nifedipine, anipamil, nicardipine, diltiazem, or a salt thereof.

5. The method of claim 1, wherein the agent is administered via oral, parenteral, subcutaneous, intradermal, intramuscular, intravenous, intraarticular, rectal, topical, or by inhalation.

6. A method of inhibiting bronchial smooth muscle remodeling in asthma as in claim 1, wherein the agent inhibits cellular signaling of calcium-dependent mitochondrial biogenesis.

7. The method of claim 6, wherein the agent inhibits expression or function of signaling molecule involved in calcium-dependent mitochondrial biogenesis.

8. The method of claim 6, wherein the inhibited signaling molecule is selected from the group consisting of mitochondrial transcription factor A, nuclear respiratory factor-1, peroxisome proliferator-activated receptor coactivator-1α, and calcium/calmodulin-dependent protein kinase IV.

9. The method of claim 6, wherein the expression or function is inhibited at the protein, DNA or RNA level.

10. The method of claim 6, wherein the agent is administered via oral, parenteral, subcutaneous, intradermal, intramuscular, intravenous, intraarticular, rectal, topical, or by inhalation.

11. The method of claim 6, wherein administration of said agent results in about 5% to about 50% decrease of asthmatic smooth muscle remodeling.

12. The method of claim 6, wherein administration of said agent results in about 5% to about 50% decrease of bronchial thickness.

13. The method of claim 6, further comprising administering to the subject one or more standard care therapy for asthma.

14. A pharmaceutical composition comprising a calcium channel blocker and a pharmaceutically acceptable vehicle, wherein the calcium channel blocker is administered in a dose sufficient to inhibit calcium-dependent mitochondrial biogenesis in asthmatic bronchial smooth muscle cells.

15. The composition of claim 14, wherein the calcium channel blocker is selected from the group consisting of gallopamil verapamil, devapamil, emopamil, nifedipine, anipamil, diltiazim, or a salt thereof.

16. The composition of claim 14, wherein the composition is in the form suitable for oral, parenteral, including subcutaneous, intradermal, intramuscular, intravenous and intraarticular, rectal and topical, including dermal, buccal, sublingual administration, or for administration by inhalation.

17. The composition according to claim 14, wherein the calcium channel blocker is administered in a dose sufficient to result in about 5% to about 50% decrease of asthmatic smooth muscle remodeling.

18. The composition according to claim 14, wherein the calcium channel blocker is administered in a dose sufficient to results in about 5% to about 50% decrease of bronchial thickness.

19. The composition according to claim 14, further comprising one or more standard care therapy for asthma.
